(19) United States
(12) Patent Application Publication
(10) Pub. No.: US 2017/0296239 A1
(43) Pub. Date: Oct. 19, 2017

(54) Pars Interarticularis Fracture Device

(71) Applicant: Nicholas Michael Cordaro, Vista, CA (US)

(72) Inventor: Nicholas Michael Cordaro, Vista, CA (US)

(73) Assignee: Additive Innovations, LLC, Carlsbad, CA (US)

(21) Appl. No.: 15/131,456

(22) Filed: Apr. 18, 2016

(51) Int. Cl. A61B 17/70; A61B 17/80; A61B 17/68

(52) U.S. Cl. CPC A61B 17/7071; A61B 17/8014; A61B 17/7059; A61B 2017/681

(57) ABSTRACT

This device is intended for orthopedic applications, particularly to treat a pars interarticularis fracture. The device is highly specialized to treat a common pars fracture with a uniquely contoured device that stabilizes the inferior fractured element with a generally conforming and non-linear hooked surface then transcribing the pars fracture to rigidly attach to the cortical bone between the facet and pedicle bones while applying compression across the fraction for fracture healing and stabilization.

### CROSS REFERENCE

Provisional Application No. 62/127,174

Under the benefit of 37 CFR 1.78, a petition to grant the filing date benefit of provisional application 62/127,174 filed on 2 Mar. 2015 is hereby respectively submitted. The delay in filing the nonprovisional application was unintentional and the required petition fee has been paid.

### STATEMENT OF FEDERALLY SPONSORED RESEARCH OR DEVELOPMENT

This application and device have received no federally sponsored research or development assistance.

### BACKGROUND

U.S. Classification 606/902, 29/592

Spondylolysis, is a condition in which a crack or fracture occurs in the posterior lateral section(s) of vertebrae of a patient's spinal column or the pars interarticularis. This spondylolysis stress fracture, commonly referred to as a pars fracture normally occurs in children and adolescents, most commonly with those who participate in activities involving repetitive motions with extension. It additionally, a pars fracture may occur in patients involved in an extension trauma event. The fracture may occur on one or both sides of a vertebra and may occur along multiple vertebrae. Current treatments are only partially effective, and many patients will experience continual low back pain, limited return to sports, and other complications throughout adulthood.

Existing initial treatments may consist of nonsteroidal anti-inflammatory medications, steroid injections, physiotherapy, bracing, and activity modification. If persistent pain after non-operative treatment continues, surgery may be required. One of the more typical operations that may be performed includes a laminectomy to remove a portion of the vertebra, including the pars fracture. This will decompress the nerve roots, resulting in short term relief, yet will destabilize the spine, likely resulting in further surgeries. A second and more common option occurring after cascading co-morbidities, includes a posterior lumbar fusion. A posterior lumbar fusion involves fusing two or more adjacent vertebral bodies, including the pars defect. This surgery will prevent further trauma to the pars defect, but will unfortunately render the operative location motionless and likely result in cascading revision surgeries for the adolescent throughout adulthood.

Other less common surgical means include fracture reduction with facet or trauma screws as well as with wires and cables. These have seen some clinical success but have not become common treatment options. For younger patients, this is not an option as there is insufficient bone for internal screw placement.

The presented invention will allow for improved surgical devices, specifically for these young patients, to allow them an opportunity for an improved physical and emotional future with a more advanced clinical fracture repair devices. Direct repair of a pars defect will allow for restoration of normal anatomy of the posterior elements, preservation of the functional motion segment, less surgical trauma with dissection restricted only lateral to the spinous process, less blood loss, and early functional recovery. The device is removable after fusion to allow for normal spinal growth after fracture repair.

As expanded indications for the device, adult and elder patients with pars fractures and instability may be treated with the device per the presented benefits.

Other means for pars fraction repair devices have been disclosed in prior art. For example, first reference, U.S. Pat. No. 7,883,532, to Robert Biscup, titled Vertebral pars interarticularis clamp a new spine fixation device, instrumentation, and methodology, refers to a hub and first and second arm connected and movable to at least partially and telescopically clamp to the lamina across a pars fracture to secure the pars fracture. The device generally traverses parallel to the pars fracture and is generally non-conforming to the anatomy. It does not compress across or perpendicular to the fracture.

A second reference, U.S. Published Pending patent application Ser. No. 13/438,912, to Carl Pasquale Giordano, titled Plates configured to rigidly fix fragments of a pars interarticularis to one another, discloses a generally contouring outer member secured to a pedicle by means of a pedicle screw that nests within the contour. The contour is open to allow for bone graft placement to the pars fracture and is generally conforming to the lamina and pedicle. The contour is secured to the lamina, inferior to the fracture, with small threaded fixation screws. This device does not teach fracture compression and relies on small bone screws to affix the device to the lamina. The pedicle screw placement presents a trajectory that may require partial removal and therefor destabilization of the adjacent facet joint while the pedicle of an adolescent may not be of sufficient geometric proportions to accept a pedicel screw.

A third reference, U.S. Published Pending patent application Ser. No. 12/561,141, to Dimitriy G. Kondrashov, titled System and method to stablize a spinal column including a spinolaminar locking plate, includes a contoured lamaina plate with threaded holes that guides and fixates threaded screws that first penetrate the spinous process, second penetrate across a facet capsule adapted to penetrate an articular process of the targeted vertebra and the mating articular process of a vertebra above the targeted vertebra to resist motion at the facet joint, and third to penetrate a pedicle or lamina. The screws additionally nest within the contoured plate. The screws are taught to penetrate across anatomical joints to fuse adjacent vertebral bodies. The Kondrashov application includes anteriorly protruding features used to stabilize the plate's placement during surgery but which do not teach a snap fit or conforming condition to the anatomy for post-surgical stabilization and which do not wrap to the anterior aspect of the lamina.

A fourth reference, U.S. Pat. No. 4,257,409, to Kazimierz Bacal, titled Device for treatment of spinal curvature, discloses hook features that wrap around to the anterior aspect of the lamina along either the superior or inferior edges of the lamina. The hook features are generally parallel to the anterior hook feature and contain constant radii, straight slopes, and parallel features that exist as straight extrusion cuts perpendicular to a single plane.

### BRIEF SUMMARY OF THE INVENTION

This device is intended for orthopedic applications, particularly to treat a pars interarticularis fracture. The device is highly specialized to treat a common pars fracture with a uniquely contoured device that stabilizes the inferior fractured element with a generally conforming and non-linear hooked surface then transcribing the pars fracture to rigidly attach to the cortical bone between the facet and pedicle bones while applying compression across the fraction for fracture healing and stabilization.

### BRIEF DESCRIPTION OF THE DRAWINGS

FIGS. 1A-1D are perspective views of common vertebra bodies and pars interarticularis fractures.

FIG. 2A-2D are perspective views of a plate configured to be attached to a pars area of a vertebra.

FIG. 3A-3B are perspective views of a plate attached to a lamina area of a vertebra.

FIG. 4A-4B are perspective views of a plate attached to a lamina area of a vertebra with a secondary lamina and primary superior screw fixation.

FIG. 5A-5B are perspective views of a plate and primary superior screw.

FIG. 6A-6C are perspective and cross-sectional views of a plate and a non-pivotally primary superior screw mating relationship.

FIG. 7A-7C are perspective and cross-sectional views of a plate and a pivotally primary superior screw mating relationship.

FIG. 8A-8C are perspective views of a snap fit plate attached to a lamina area of a vertebra across a pars fracture.

### DETAILED DESCRIPTION OF THE INVENTION

A novel device and method for fracture fixation of a defect seen within the pars interarticularis (pars) is disclosed.

There are three key advanced technologies, that when used in collaboration, have allowed the invention the ability to treat pars fractures with a highly focused device and method. These include more accurate computer tomography and magnetic resonance scans and datasets, 3-D computational reconstructions, and additive manufacturing. The more accurate imaging systems have allowed for a proper diagnosis and cataloging of the defect. The 3-D computational reconstructions have allowed for the system to be created around common anatomical fractures while offering more specific geometries of less common anatomical pathologies. Additive manufacturing (titanium DMLS) has allowed for a cost effective means to commercialize the pars fracture repair plates per the complex geometry. Additive manufacturing of metals such as titanium and stainless, plastics such as PEEK and PEAK, restorable polymers, and collagen, or including any combination thereof are included within the scope of this invention. Traditional subtractive manufacturing, molding, and forming is additionally included within the scope of this invention. Post treatments such as tumbling, polishing, anodizing, cryo-deburr, HA surface treatments, and nano surfaces may further be included.

The physical devices disclosed are fusion plates forming a more complex and non-linear mating surface profile as required to match the defect zone. A novel feature of this invention is that the plate will additionally wrap around the boney structures sufficiently to stabilize the fracture without the placement of secondary bone screws. However this secondary bone screw will allow for further stabilization to the lamina as well as to allow for secondary device attachment, such as to a rod, tulip head, connector, or other means. The primary bone screw as later disclosed, acts across the fracture in a generally perpendicular manner as to present at least a partial force vector to compress the presented fracture, without protruding into or in a manner locking or restricting motion of a facet capsule. Partial motion restrictions, depending on patient anatomy and final device placement, may be restricted as part of post-surgical management or until the device is removed once the fracture has healed.

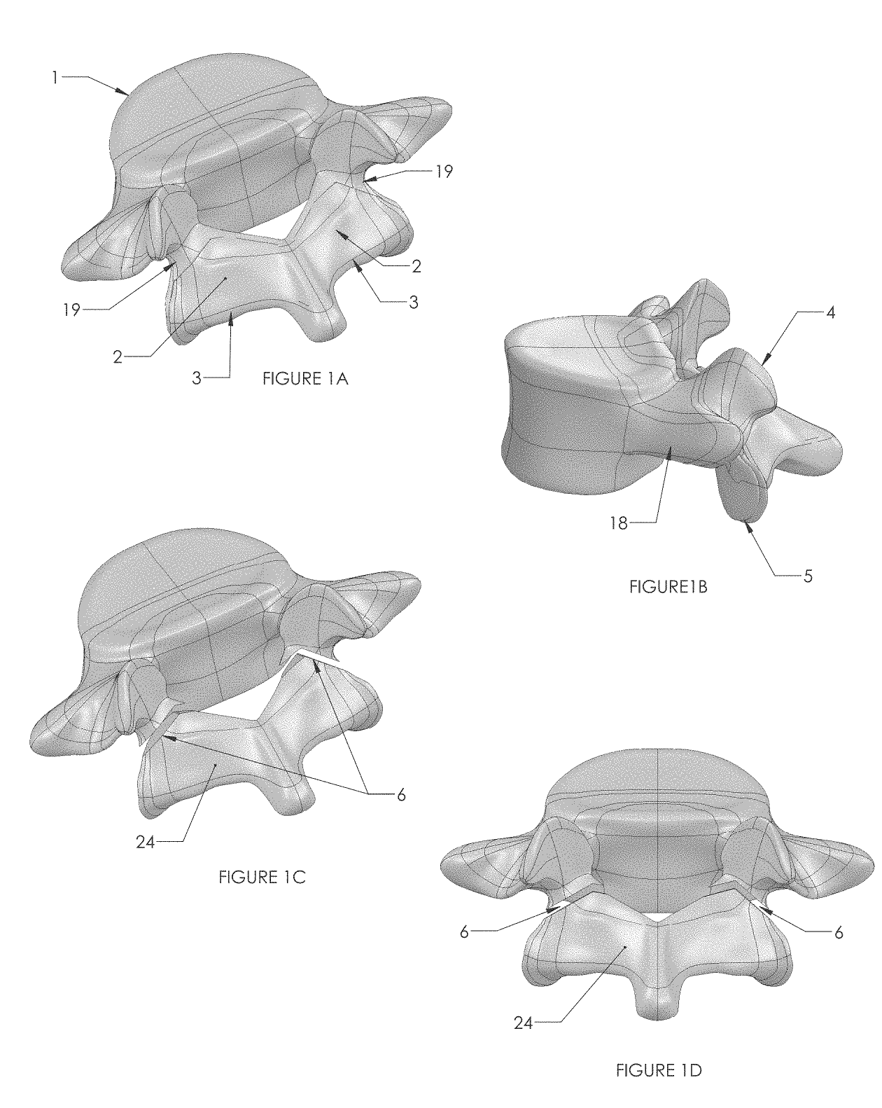

The preferred embodiment of the plate being disclosed is designed to interact with features of a vetebral body 1 shown in FIGS. 1A and 1B. FIGS. 1C and 1D illustrate a form of fracture 6 known to occur in a region of the vertebrae 1 known as the pars interarticularis 19. This fracture 6 may occur on one or both of the pars interarticularis 19.

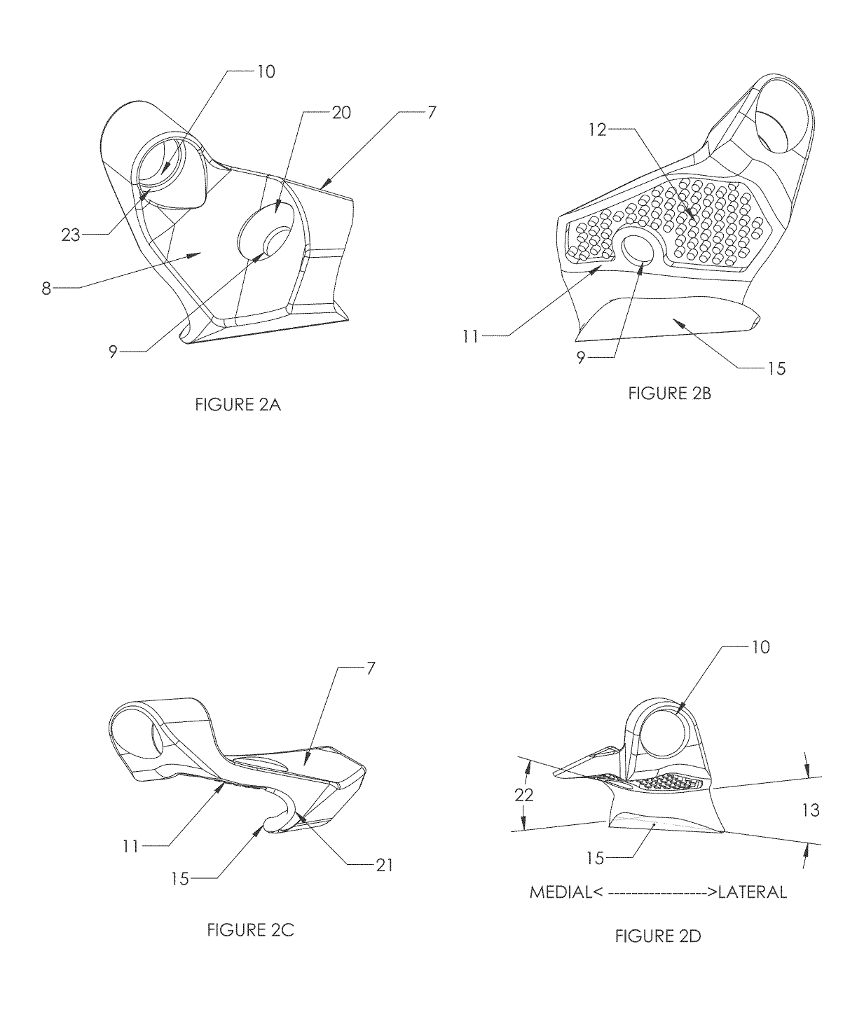

Referring FIGS. 2A through D, the preferred embodiment of the plate 7 has a posterior superior surface 8 and an anterior inferior surface 11. The plate 7 has a location for a single cortical screw 16 for placement 9 into the base of the spinous process at the intersection of the lamina 3, securing the pars plate to the fractured side of the defect 24. This screw will help reduce the fracture to the plate. The cortical screw location 9 has a countersink chamfered edge 20 on the superior surface 8 of the plate which allows the head of the cortical screw 16 to sit flush with the plate surface 8 when properly placed. The cortical screw may be replaced or amended with a member to rigidly join to an adjacent plate.

The plate also contains a single second screw location 10 positioned across or in proximity to the fracture with a trajectory between the superior 4 and inferior 5 articular processes for plate securement on the opposite side of the fracture.

Pockets or features 12 of varying shape or porosity on the inferior surface 11 of the plate to induce osteoconduction or for which to secure the graft material such as but not limited to autograft, allograft, BMP, etc., creating a more secure bond between the plate 7, vertebral lamina 2 and across the fracture 6. The plate 7 also has a hook feature 15 which will engage the inferior edge 3 of the lamina.

Of additional importance to the hook feature 15 is that is flairs outward as it travels lateral 13. If the cortical screw 16 is placed last, the hook feature will tighten against the boney anatomy as the lag screw 17 is tightened, pulling the plate across the fracture and slightly lateral into a tighter seating position.

A similar flaring will be taught on the medial hook aspect 22, as shown in FIG. 2D. This feature offers clearance to the boney anatomy at the intersection to the spinous process and may also further seat as the lag screw is advanced. These flaring features may be linear or non-linear and of constant or variable radii.

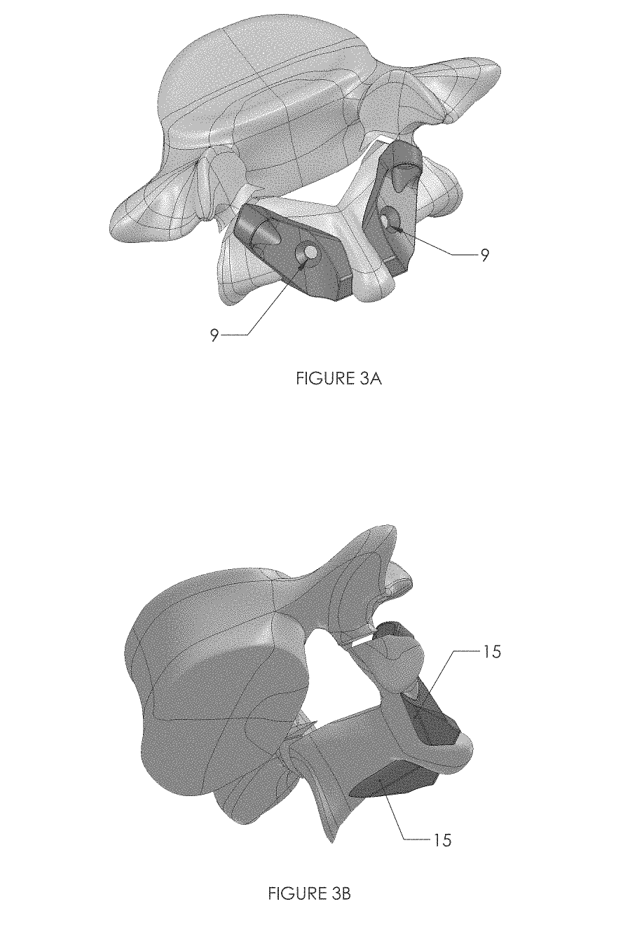

Referring to FIGS. 3A and B, the plate 7 is shown placed on the lamina 3 of the vertebrae 1. Location for placement 9 of the cortical screw 16 into the lamina is readily apparent. Additionally, the lamina hook 15 is engaged to the inferior edge of the vertebral lamina 3 and may curve slightly to the anterior surface of the lamina.

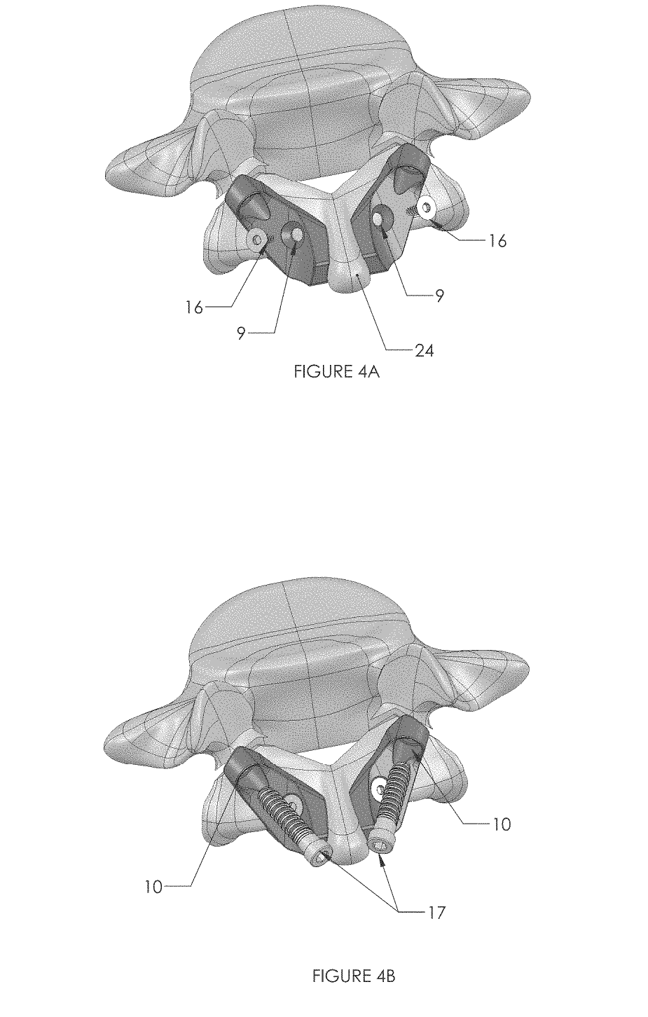

FIG. 4A, illustrates placement of the cortical screw 16 through the plate 7 securing it to the fractured side of the defect 24.

FIG. 4B illustrates placement of the lag screw 17 through the plate 7.

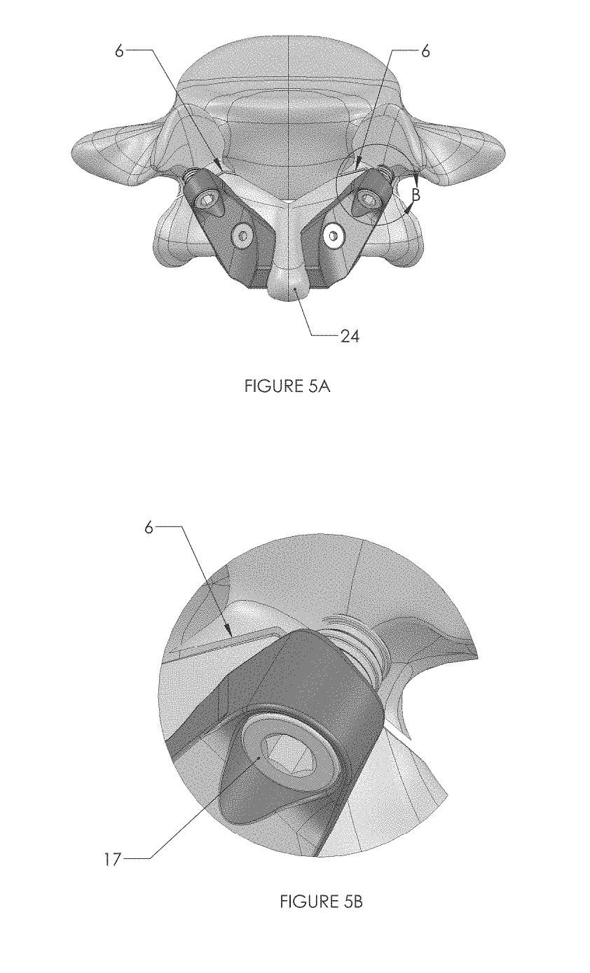

As shown in FIGS. 5A and 5B reduction of the fracture 6 is accomplished by placement of the lag screw 17. The lag screw 17 is fully seated in the boney anatomy of the vertebrae 1.

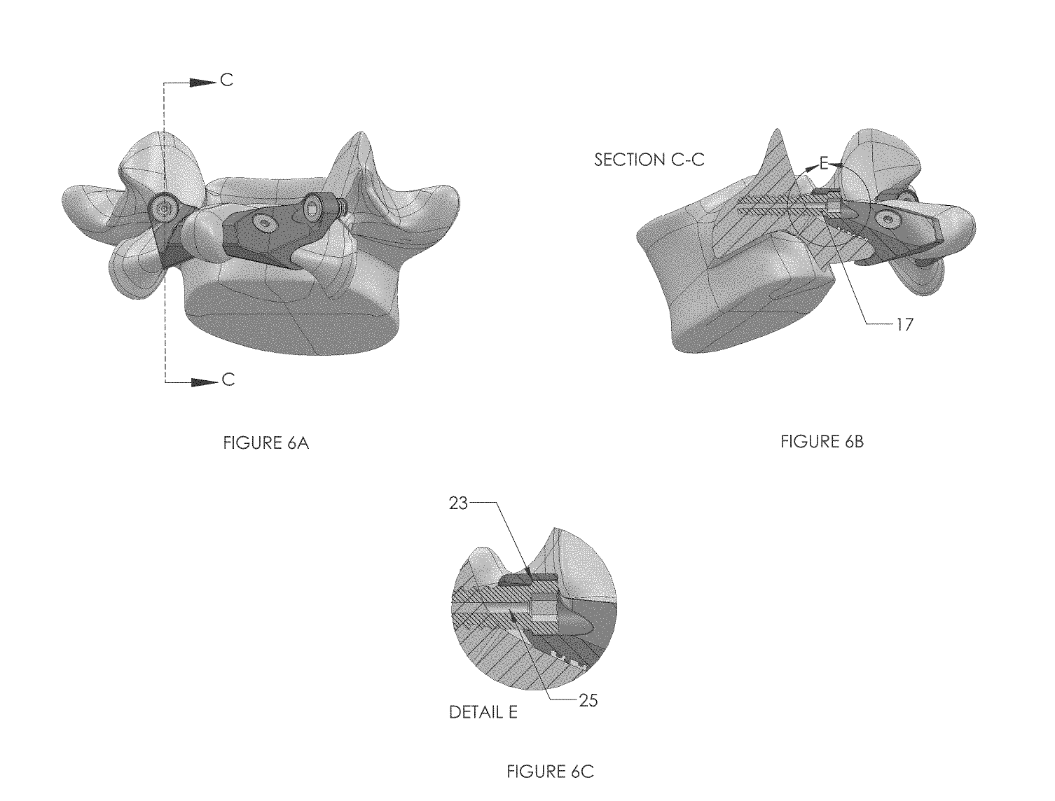

FIGS. 6A, B and C illustrate features of the lag screw 17 and plate 7 designed to facilitate placement and reduction of the fracture 6. The lag screw 17 may have a cannulation 25 to facilitate use of a guide wire during placement. Reduction of the fracture 6 is accomplished by interface with an internal mating chamfer 23 on the plate.

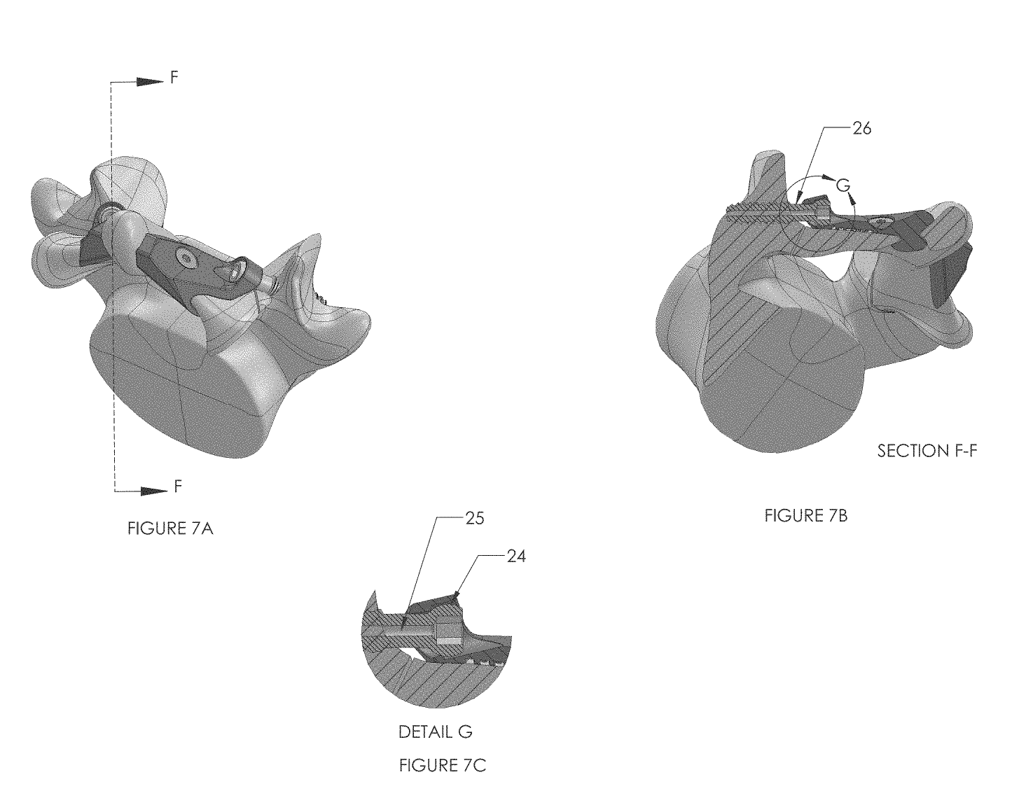

As shown in FIGS. 7A, B and C, the lag screw 26, may have a spherical head 24 which allows for greater variety in the angle of placement of the screw into the vertebrae 1. The spherical headed lag screw may also have a cannulation 25 to facilitate use of a guide wire during placement.

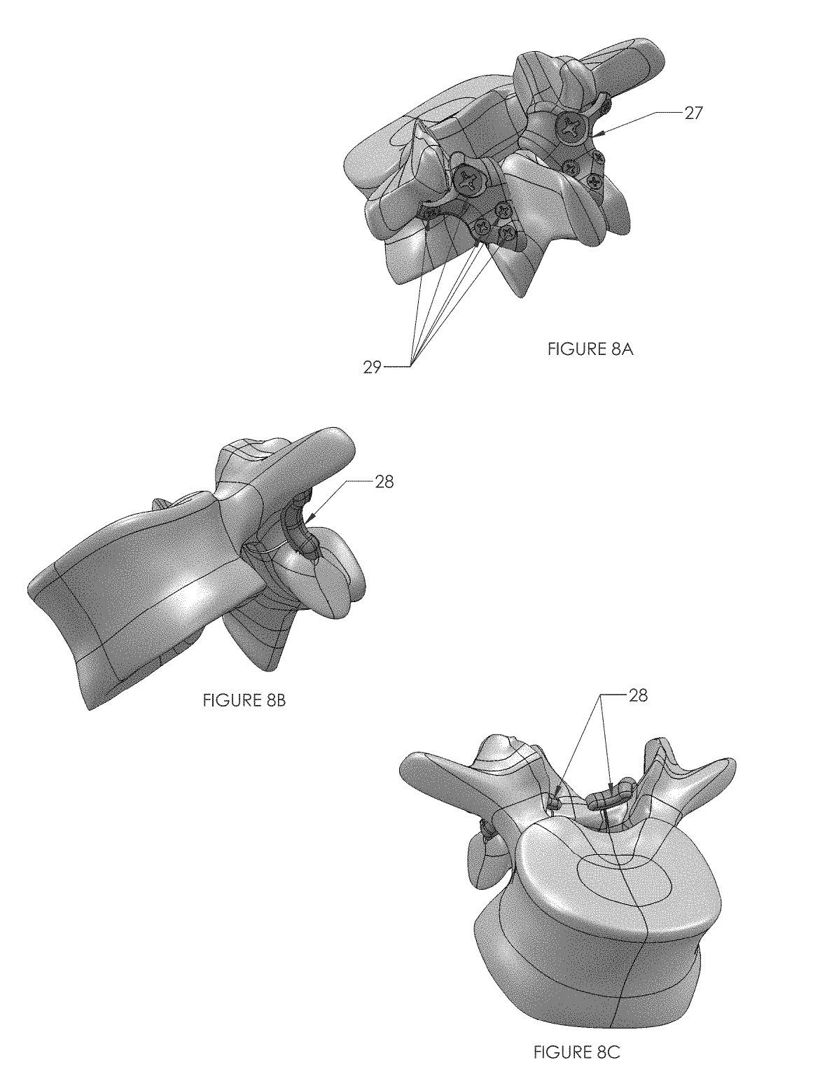

As shown in FIGS. 8A, B and C, another embodiment of the device 27 may have features 28 which wrap around the bone structures and will not remain on the posterior aspect of the pars and adjacent boney structures (including the posterior lateral) alone. The presented plate will snap onto the boney structures and secure the fracture. Screws 29 will further stabilize the system for fracture healing but are not required to transverse the pedicle for stability

1. A pars interarticularis fracture fixation plate comprising:
A first section intended to be fixed inferior to the superior articular process and second section medial to the inferior articular process and a third section contoured to the lamina and connecting the said first and second sections in which the first section includes a non-threaded fixation pathway and threaded member elevated, and with a non-parallel surface to the third section and in which the second section includes an angled inferior opening to capture the inferior aspect of the lamina extending to the anterior aspect of the lamina.

2. The system of claim 1, further comprising:
Mating surfaces between the non-treaded fixation pathway and the threaded member to where the threaded member may freely rotate within the fixation pathway yet configured to prevent the threaded member from fully translating through the non-threaded fixation pathway.

3. The system of claim 2, further comprising:
A rotatable threaded member wherein when rotated into the boney structures anterior to the superior facet capsule will advance the plate creating at least one force vector compressing and acting perpendicular to the pars fracture for fracture reduction.

4. The system of claim 3, further comprising:
Mating surfaces between the non-treaded fixation pathway and the threaded member to where the threaded member may freely rotate and pivot within the fixation pathway.

5. The system of claim 3, further comprising:
Mating surfaces between the non-treaded fixation pathway and the threaded member to where the threaded member may freely rotate but present limited to no ability to pivot within the fixation pathway forcing a co-linear relationship.

6. The system of claim 1, further comprising:
A third section containing a second fixation pathway adapted to receive a second screw to fix the lamina or the base of the spinous process to the third section.

7. The system of claim 6, further comprising:
A third section containing a second fixation pathway adapted to receive a member used to fix the plate to an adjacently placed fixation means.

8. A pars interarticularis fracture fixation plate comprising:
A first section intended to be fixed inferior to the superior articular process and second section medial to the inferior articular process and a third section contoured to the lamina and connecting the said first and second sections in which the second section includes an angled inferior opening to capture the inferior aspect of the lamina extending to the anterior aspect of the lamina in which the anterior aspect of the second section is non parallel to the posterior section of the second section.

9. The system of claim 8, further comprising:
An anterior aspect of the second section that is conforming and curved to the anterior aspect of the lamina with one or more radii.

10. The system of claim 8, further comprising:
An anterior aspect of the third section that is conforming and curved to the anterior aspect of the lamina with one or more variable radii.

11. The system of claim 8, further comprising:
An anterior aspect of the third section that is conforming and curved to the anterior aspect of the lamina with one or more variable surfaces.

12. A pars interarticularis fracture fixation plate comprising:
A first section intended to be fixed inferior to the superior articular process and second section medial to the inferior articular process and a third section contoured to the lamina and connecting the said first and second sections in which the second section includes an inferior opening to capture the inferior aspect of the lamina extending to the anterior aspect of the lamina in which the anterior opening aspect of the second section is skewed to the posterior opening aspect of the second section.

13. A pars interarticularis fracture fixation plate comprising:
A first section intended to be fixed inferior to the superior articular process with a compressive lag screw and second section medial to the inferior articular process and a third section connecting the said first and second sections in which the second section includes an inferior opening to capture the inferior aspect of the lamina extending to the anterior aspect of the lamina in which the anterior opening aspect of the second section is skewed to the posterior opening aspect of the second section.
